(19) United States
(12) Patent Application Publication
(10) Pub. No.: US 2026/0315426 A1
(43) Pub. Date: Oct. 15, 2026

(54) USE OF INGENOL MEBUTATE OR A COMPOSITION COMPRISING THE SAME IN THE PREPARATION OF A MEDICAMENT FOR THE TREATMENT OF TUMORS

(71) Applicant: Affiliated Hospital of Nantong University, Nantong (CN)

(72) Inventors: Bo YOU, Nantong (CN); Tian XIA, Nantong (CN); Yiwen YOU, Nantong (CN); Kaiwen ZHANG, Nantong (CN); Ying SHAN, Nantong (CN)

(73) Assignee: Affiliated Hospital of Nantong University, Nantong (CN)

(21) Appl. No.: 19/411,322

(22) Filed: Dec. 7, 2025

(30) Foreign Application Priority Data
Apr. 9, 2025 (CN) 202510440536.3

(51) Int. Cl. A61K 31/215; A61K 39/395; A61P 35/00

(52) U.S. Cl. CPC A61K 31/215; A61K 39/3955; A61P 35/00

(57) ABSTRACT

A use of ingenol mebutate (IM) or a composition including the same in the preparation of pharmaceutical agents for tumor treatment pertain to the field of biomedicine. IM can inhibit the formation of vasculogenic mimicry (VM) following treatment with anti-angiogenic medicament. When used in combination with anti-angiogenic drugs (AADs) such as nimotuzumab (NTZ), IM inhibits the development of drug resistance to AADs, further reducing the recurrence and metastasis of nasopharyngeal carcinoma (NPC). Thereby, it enhances the survival rate and improves the prognosis of NPC patients. The disclosure has significant clinical implications and strong potential for industrial application and commercialization.

### CROSS-REFERENCE TO RELATED APPLICATION

This application claims priority to Chinese Patent Application No. 202510440536.3 filed on Apr. 9, 2025, the disclosure of which is hereby incorporated by reference in its entirety.

### BACKGROUND

In recent years, a variety of anti-angiogenic drugs (AADs) have been approved by regulatory authorities for the clinical treatment of solid tumors, including nasopharyngeal carcinoma (NPC). Studies have demonstrated that anti-angiogenic therapies (AATs) can significantly improve key clinical indicators such as progression-free survival and disease-free survival in patients. However, certain limitations have also been exposed in the treatment of malignant tumors. Although certain efficacy is observed during the initial treatment phase, the development of drug resistance is inevitable, and a significant improvement in overall patient survival has not been achieved. Research into drug resistance mechanisms reveals that vascular remodeling plays a critical role in resistance to AATs, and the formation of vasculogenic mimicry (VM) represents one of its key mechanisms. VM, as a novel tumor blood perfusion system, is composed of highly malignant tumor cells and basement membrane, functioning entirely independently of the traditional endothelial-dependent angiogenesis pathway. VM not only exhibits the functions of a mature microcirculation but is also significantly associated with the metastatic potential and adverse prognosis of various highly aggressive malignant tumors. Existing AADs, due to their limited targets, are unable to effectively inhibit the formation of VM. Currently, there is a clinical lack of therapies specifically targeting VM to overcome resistance to AADs, inhibit recurrence and metastasis of malignant tumors, and improve patient prognosis.

In view of the above, the present disclosure provides a use of IM or a composition comprising the same in the preparation of a medicament for the treatment of tumors.

### SUMMARY

In order to address, at least in part, the technical problems described above, embodiments of this disclosure provide a use of ingenol mebutate (IM) or a composition comprising the same in the preparation of a medicament for the treatment of tumors. The specific technical solutions are as follows:

The present disclosure relates to the field of biomedical technology, and more particularly to a use of IM or a composition comprising the same in the preparation of a medicament for the treatment of a tumor.

In a first aspect of this disclosure, provided is a use of IM in the preparation of a medicament for the treatment of a tumor, wherein the medicament inhibits the formation of VM in said tumor. The molecular formula of IM is C₂₅H₃₄O₆, and the structural formula of IM is represented by the following formula:

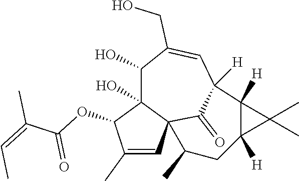 [SMILES: C/C=C(/C)C(=O)O[C@H]1C(C)=C[C@]23C(=O)[C@@H](C=C(CO)[C@@H](O)[C@]12O)[C@H]1[C@@H](C[C@H]3C)C1(C)C]

The promotion of microcirculatory network formation in solid cancers through VM is a phenomenon prevalent in solid tumors such as glioma, melanoma, and breast cancer, among others, and is not cancer-type specific. This disclosure has discovered that IM can effectively inhibit the formation of VM, and thus can be extended for application in the treatment of other malignant cancers.

In a second aspect of this disclosure, provided is a use of a combined pharmaceutical composition in the preparation of a medicament for the treatment of a tumor, wherein the combined pharmaceutical composition comprises an AAD and IM.

In some embodiments of this disclosure, the AAD is nimotuzumab (NTZ).

In some embodiments of this disclosure, in the combined pharmaceutical composition, the mass ratio of the AAD to IM is 1:1.

In some embodiments of this disclosure, the medicament for treating tumors inhibits tumor metastasis and/or recurrence.

In some embodiments of this disclosure, the combined pharmaceutical composition is a combination of two separate formulations, wherein the two separate formulations are administered simultaneously or sequentially.

In some embodiments of this disclosure, the medicament for treating tumors further comprises a pharmaceutically acceptable excipient.

In some embodiments of this disclosure, the aforementioned tumor is NPC.

Compared with the prior art, this disclosure provides the following technical effects:

This disclosure discovers that IM, by inhibiting the formation of VM during AATs for malignant tumors, can synergize with AADs to provide an improved clinical treatment protocol for cancer, particularly NPC. Experimental results demonstrate that the advantages include: (1) IM inhibits the formation of VM; and (2) the combination of IM with an AAD significantly reduces tumor (e.g., NPC) metastasis and markedly improves prognosis.

### BRIEF DESCRIPTION OF THE DRAWINGS

For a clearer illustration of the technical solutions in the embodiments of this disclosure or in the prior art, the drawings to be used in the description of the embodiments or the prior art will be briefly introduced below.

FIG. 1A shows images of CD31/PAS immunohistochemical double staining in paraffin-embedded sections of in situ tumors from patients with NPC at different clinical stages;

FIG. 1B shows a statistical graph of VM and MV numbers in paraffin-embedded sections of in situ tumors from early-stage (Clinical Stage 1-2) and late-stage (Clinical Stage 3-4) NPC patients;

FIG. 1C shows the survival curves of NPC patients based on the number of VM or MV in paraffin-embedded sections of in situ NPC tumors;

FIG. 2A shows images of CD31/PAS immunohistochemical double staining in paraffin-embedded sections of in situ NPC tumors from nude mice in Control Group 2 and NTZ Group 2 at different time points after inoculation;

FIG. 2B shows a statistical graph of VM, MV, and MV+VM numbers in paraffin-embedded sections of in situ NPC tumors from nude mice in Control Group 2 and NTZ Group 2 at different time points after inoculation;

FIG. 3A shows optical micrographs of VM formation in an in vitro assay for Control Group 3, Metastasis-exo Group 3, IM Group 3, and Metastasis-exo+IM Group 3;

FIG. 3B shows a statistical graph quantifying the number of VM formation in Control Group 3, Metastasis-exo Group 3, IM Group 3, and Metastasis-exo+IM Group 3;

FIG. 4A shows in vivo bioluminescence imaging representations for Control Group 4, NTZ Group 4, IM Group 4, and NTZ+IM Group 4;

FIG. 4B shows a line graph of the non-metastasis rate for Control Group 4, NTZ Group 4, IM Group 4, and NTZ+IM Group 4;

FIG. 4C shows a survival curve graph for Control Group 4, NTZ Group 4, IM Group 4, and NTZ+IM Group 4;

FIG. 4D shows representative images of CD31/PAS immunohistochemical double staining in paraffin-embedded sections of in situ NPC tumors from nude mice in Control Group 4, NTZ Group 4, IM Group 4, and NTZ+IM Group 4;

FIG. 4E shows a bar graph of the quantified counts of MV, VM, and MV+VM in tissue sections of in situ NPC tumors from nude mice in Control Group 4, NTZ Group 4, IM Group 4, and NTZ+IM Group 4.

### DETAILED DESCRIPTION

A person skilled in the art may adapt the content described herein and make appropriate modifications of process parameters. It is specifically pointed out that all similar substitutions and modifications would be obvious to those skilled in the art and are considered to be included within the scope of the present disclosure. The methods and applications of this disclosure have been described through preferred embodiments. It will be apparent to relevant skilled persons that, without departing from the spirit and scope of this disclosure, modifications, appropriate variations, and combinations of the methods and applications described herein may be made to implement and apply the technology of this disclosure.

In this disclosure, all raw materials and reagents are commercially available.

### Preparation of Samples

Paraffin-embedded sections of 46 NPC tissue samples were prepared by the Department of Pathology, Affiliated Hospital of Nantong University. Pathological diagnosis and staging of the samples were performed according to the AJCC Cancer Staging Manual (8th Edition, 2018). The cohort comprised 15 patients with early-stage NPC (Clinical Stage 1-2) and 31 patients with late-stage NPC (Clinical Stage 3-4).

### Example 1

To investigate the correlation between clinical stage of NPC and the number of MV and VM in paraffin-embedded sections of in situ tumor tissues, the aforementioned 46 paraffin-embedded tissue samples were subjected to CD31/PAS immunohistochemical double staining, comprising the following steps:

(1) Conventional deparaffinization and rehydration of paraffin-embedded sections were performed, followed by antigen retrieval. An appropriate amount of endogenous peroxidase blocker was added and incubated at room temperature for 10 minutes, followed by washing with PBS (3 minutes×3 times).

(2) Primary antibody application: An appropriate amount of primary CD31 antibody (1:200 dilution) was added according to tissue size and incubated at 4° C. overnight. The sections were then washed with PBS buffer (3 minutes×3 times).

(3) An appropriate amount of enzyme-labeled goat anti-mouse/rabbit IgG polymer was added and incubated at room temperature for 20 minutes, followed by washing with PBS buffer (3 minutes×3 times).

(4) Color development: An appropriate amount of freshly prepared DAB chromogenic solution was added and incubated at room temperature for 2-5 minutes.

(5) PAS staining: The samples were rinsed with tap water for 2-3 minutes, followed by two washes with distilled water; placed in oxidizing agent at room temperature (25-30° C.) for 5-8 minutes (generally not exceeding 10 minutes); rinsed once with tap water and twice with distilled water; immersed in Schiff Reagent and stained in a dark place at room temperature for 10-20 minutes; finally rinsed with tap water for 10 minutes.

(6) The samples were placed in hematoxylin staining solution for 1-2 minutes for nuclear staining, differentiated in acid differentiation solution for 2-5 seconds, rinsed with tap water for 10-15 minutes, washed with double-distilled water to restore the blue color, dehydrated through a conventional ethanol gradient, cleared in xylene, and mounted with neutral gum.

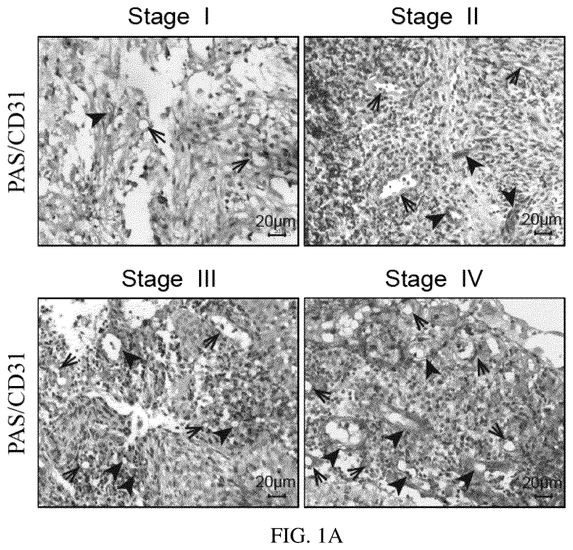

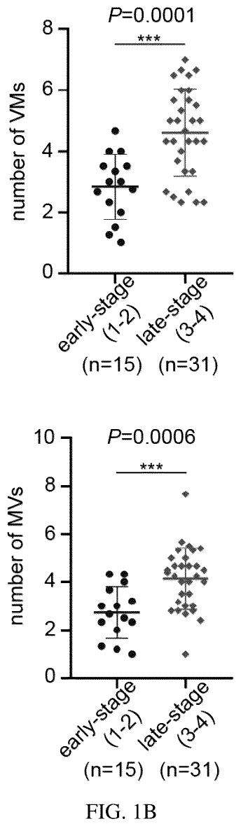

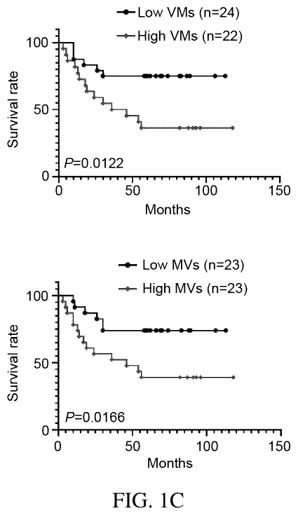

FIG. 1A is representative images of CD31/PAS immunohistochemical double staining in paraffin-embedded sections of in situ tumors from patients with NPC at different clinical stages.

Herein, CD31-PAS+structures indicate VM (indicated by normal arrows), while CD31+PAS+structures indicate MV (indicated by black solid arrows).

The numbers of VM and MV in paraffin-embedded sections of in situ tumors from early-stage (Clinical Stage 1-2) and late-stage (Clinical Stage 3-4) NPC patients were statistically analyzed, and the results are shown in FIG. 1B. FIG. 1B demonstrates that the numbers of both VM and MV in in situ tumors of early-stage NPC patients were lower than those in late-stage NPC patients.

Furthermore, based on the Kaplan-Meier method, the relationship between the number of VM or MV in the in situ tumors of NPC patient and their prognosis was analyzed. Survival curves for NPC patients with varying numbers of VM or MV in paraffin-embedded sections of the in situ tumor are depicted in FIG. 1C.

FIG. 1A to 1C collectively demonstrates that the number of VM and MV in paraffin-embedded sections of in situ tumors from late-stage NPC patients is higher than those from early-stage NPC patients. Moreover, NPC patients with a higher number of MV or VM exhibit worse prognosis compared to those with a lower number of MV or VM.

### Example 2

Dilute Nimotuzumab (NTZ) into PBS to prepare an NTZ injection. The NTZ was administered at a dosage of 20 mg per kilogram of body weight.

Control Group 2: Male BALB/c nude mice, 6 weeks old, weighing 20±5 g, were used. The mice were deeply anesthetized using chloral hydrate (4% solution, 400 mg/kg) and placed in a stereotactic frame in a supine position. The mouth was opened, the tongue was pulled laterally using curved forceps, and a 50 μL cell suspension containing 2×10⁶ luciferase-labeled CNE-2 cells was injected into the junction of the soft and hard palate using a 1 mL sterile syringe. On day 3 after tumor inoculation, the control treatment was initiated, with intraperitoneal injections of 200 μL PBS administered twice per week.

NTZ Group 2: Male BALB/c nude mice, 6 weeks old, weighing 20=5 g, were used. The mice were deeply anesthetized using chloral hydrate (4% solution, 400 mg/kg) and placed in a stereotactic frame in a supine position. The mouth was opened, the tongue was pulled laterally using curved forceps, and a 50 μL cell suspension containing 2×10⁶ luciferase-labeled CNE-2 cells was injected into the junction of the soft and hard palate using a 1 mL sterile syringe. On day 3 after tumor inoculation, the drug treatment was initiated, with intraperitoneal injections of 200 μL NTZ formulation administered twice per week.

Whole-body bioluminescence imaging (BLI) combined with histological examination of paraffin-embedded sections was employed to monitor the growth of primary NPC tumors and the metastasis of tumors away from the primary NPC site. The results are shown in Table 1. The metastasis rate was calculated according to the following formula:

Metastasis Rate (%)=(Number of mice with metastasis/Total number of mice)×100%

TABLE 1
Statistics of metastasis in the clinically highly
relevant orthotopic NPC metastasis model using
nude mice in Control Group 2 and NTZ Group 2.

| | Number | Number | | |
|---|---|---|---|---|
| | of mice | of mice | | |
| | with | without | Total | Metastasis |
| Group | metastasis | metastasis | number | Rate (%) |
| Control group 2 | 5 | 7 | 12 | 41.7 |
| NTZ group 2 | 6 | 9 | 15 | 40 |

The difference in metastasis rates between the two groups was analyzed using the chi-square test. The results showed no statistically significant difference (p>0.05), indicating that NTZ treatment did not inhibit NPC metastasis.

On days 5, 8, 13, and 18 post-tumor inoculation, three nude mice were euthanized respectively. Their in situ tumors were embedded in paraffin and subjected to CD31/PAS immunohistochemical double staining following the same method as described in Example 1. The numbers of MV and VM in the sectioned samples were recorded.

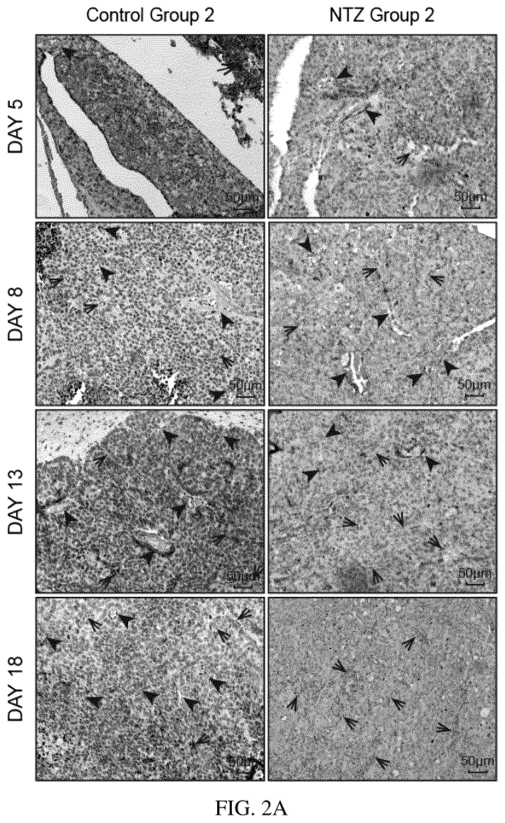

FIG. 2A is representative images of CD31/PAS immunohistochemical double staining of paraffin-embedded tissue sections from NPC in situ tumors in nude mice at different time points after inoculation for Control Group 2 and NTZ Group 2. Herein, CD31-PAS+structures (luminal pink staining) indicate VM (indicated by normal arrows), while CD31+PAS+ structures (luminal mixed brownish-tan and pink staining) indicate MV (indicated by black solid arrows).

FIG. 2B is a statistical line graph of VM, MV, and MV+VM numbers in paraffin-embedded sections from NPC in situ tumors in nude mice at different time points after inoculation for Control Group 2 and NTZ Group 2; As demonstrated in FIGS. 2A and 2B, NTZ treatment, while inhibiting intratumoral MV, resulted in a compensatory increase in VM. Consequently, the treatment did not demonstrate a significant suppression of the total microcirculation capacity (MV+VM) within the tumors.

### Example 3

Exosomes were isolated from 5 mL of peripheral blood obtained from patients with metastatic NPC via gradient centrifugation. The isolated exosomes were resuspended in 100 μL of 1640 medium to obtain metastasis-exos suspension. 50 μL of metastasis-exos suspension was added to every 10 mL of 1640 medium supplemented with 10% fetal bovine serum (FBS) to obtain 1640 medium containing metastasis-exos.

IM was dissolved in dimethyl sulfoxide (DMSO) and subsequently diluted into 1640 medium supplemented with 10% FBS to obtain IM-containing 1640 medium, wherein the concentration of IM was 5 μM.

In the 1640 medium containing metastasis-exos, add the IM solution dissolved in DMSO to achieve a final IM concentration of 5 μM, resulting in 1640 medium containing IM plus metastasis-exos.

Control Group 3: Cell lines of NPC (CNE-2, C666-1, and HK1) were seeded in 6-well plates and cultured for 1 day.

Metastasis-exos Group 3: Cell lines of NPC (CNE-2, C666-1, and HK1) were seeded in 6-well plates and cultured for 1 day, wherein the culture medium was then replaced with the 1640 medium containing metastasis-exos followed by an additional 1 day of culture.

IM Group 3: Cell lines of NPC (CNE-2, C666-1, and HK1) were seeded in 6-well plates and cultured for 1 day, wherein the culture medium was then replaced with IM-containing 1640 medium followed by an additional 1 day of culture.

IM+Metastasis-exos Group 3: Cell lines of NPC (CNE-2, C666-1, and HK1) were seeded in 6-well plates and cultured for 1 day, wherein the culture medium was then replaced with the 1640 medium containing IM plus metastasis-exos followed by an additional 1 day of culture.

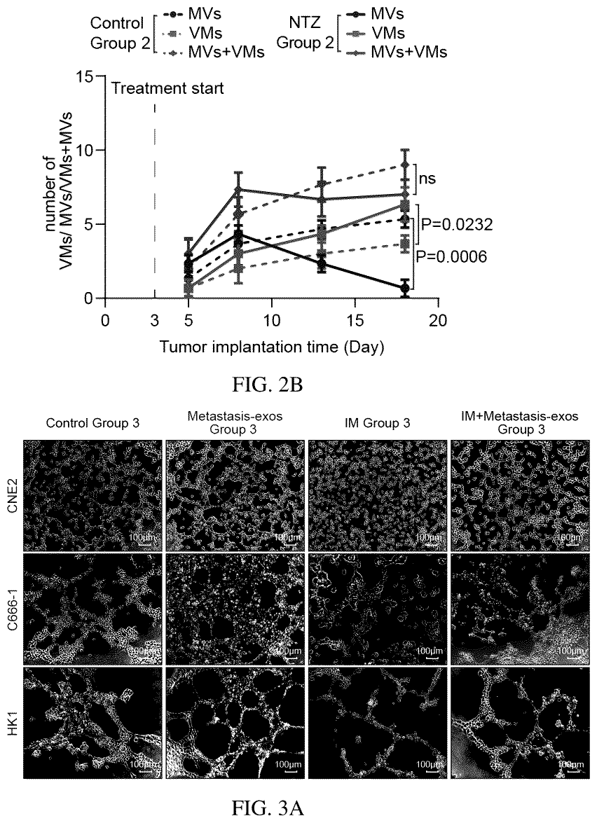

A matrigel mixture was prepared by combining Matrigel with 1640 medium at 1:1 ratio, followed by thorough mixing. The mixture was added to a 96-well plate at 50 μL per well and incubated at 37° C. for 30 minutes to allow solidification. Cells from Control Group 3, Metastasis-exos Group 3, IM Group 3, and IM+Metastasis-exos Group 3 were digested. Subsequently, 3×10⁴ cells from each group were seeded per well onto the solidified matrigel mixture and cultured for 6-8 hours. The results were observed under an optical microscope, as shown in FIG. 3A. The number of VM formed by cells from Control Group 3, Metastasis-exos Group 3, IM Group 3, and IM+Metastasis-exos Group 3 on the matrigel mixture was statistically analyzed. The results are presented in FIG. 3B. As demonstrated in FIGS. 3A and 3B, metastasis-exos promoted the formation of VM, whereas IM demonstrated a significant inhibitory effect on VM formation. Furthermore, IM reversed the VM-promoting effect induced by metastasis-exos.

### Example 4

NTZ was diluted in PBS to prepare an NTZ injection. The NTZ was administered at a dosage of 20 mg per kilogram of body weight.

IM was dissolved in DMSO and subsequently diluted into PBS to prepare an IM injection. The IM was administered at a dosage of 20 mg per kilogram of body weight.

IM was dissolved in DMSO and subsequently diluted into PBS. NTZ was then added to the solution to prepare NTZ+IM injection. In the resulting injection, NTZ and IM were present in a mass ratio of 20 mg to 20 mg.

Control Group 4: Male BALB/c nude mice, 6 weeks old, weighing 20±5 g, were deeply anesthetized with chloral hydrate (4% solution, 400 mg/kg) and placed in a stereotactic frame in the supine position. The mouth was opened, the tongue was pulled laterally using curved forceps, and a 50 μL cell suspension containing 2×10⁶ luciferase-labeled CNE-2 cells was injected into the junction of the soft and hard palate using a 1 mL sterile syringe. Control treatment was initiated on day 4 post-tumor inoculation, with intraperitoneal injections of 200 μL PBS administered twice weekly.

NTZ Group 4: Male BALB/c nude mice, 6 weeks old, weighing 20=5 g, were deeply anesthetized with chloral hydrate (4% solution, 400 mg/kg) and placed in a stereotactic frame in the supine position. The mouth was opened, the tongue was pulled laterally using curved forceps, and a 50 μL cell suspension containing 2×10⁶ luciferase-labeled CNE-2 cells was injected into the junction of the soft and hard palate using a 1 mL sterile syringe. Drug treatment was initiated on day 4 post-tumor inoculation, with intraperitoneal injections of 200 μL NTZ formulation administered twice weekly.

IM Group 4: Male BALB/c nude mice, 6 weeks old, weighing 20=5 g, were deeply anesthetized with chloral hydrate (4% solution, 400 mg/kg) and placed in a stereotactic frame in the supine position. The mouth was opened, the tongue was pulled using curved forceps, and a 50 μL cell suspension containing 2×10⁶ luciferase-labeled CNE-2 cells was injected into the junction of the soft and hard palate using a 1 mL sterile syringe. Drug treatment was initiated on day 4 post-tumor inoculation, with intraperitoneal injections of 200 μL IM formulation administered twice weekly.

NTZ+IM Group 4: Male BALB/c nude mice, 6 weeks old, weighing 20=5 g, were deeply anesthetized with chloral hydrate (4% solution, 400 mg/kg) and placed in a stereotactic frame in the supine position. The mouth was opened, the tongue was pulled using curved forceps, and a 50 μL cell suspension containing 2×10⁶ luciferase-labeled CNE-2 cells was injected into the junction of the soft and hard palate using a 1 mL sterile syringe. Drug treatment was initiated on day 4 post-tumor inoculation, with intraperitoneal injections of 200 μL NTZ+IM formulation administered twice weekly.

Whole-body BLI combined with histological examination of paraffin-embedded sections was employed to monitor the growth of primary NPC tumors and the metastasis of to distant sites. The results are shown in Table 2. The metastasis rate was calculated according to the following formula:

Metastasis Rate (%)=(Number of mice with metastasis/Total number of mice)×100%

TABLE 2
Statistics of metastasis in the clinically highly relevant
orthotopic NPC metastasis model using nude mice in Control
Group 4, NTZ Group 4, IM Group 4, and NTZ + IM Group 4.

| | Number | Number | | |
|---|---|---|---|---|
| | of mice | of mice | | |
| | with | without | Total | Metastasis |
| Group | metastasis | metastasis | number | Rate (%) |
| Control group 4 | 8 | 7 | 15 | 53.3 |
| NTZ group 4 | 6 | 9 | 15 | 40 |
| IM group 4 | 5 | 10 | 15 | 33.3 |
| NTZ + IM group 4 | 1 | 14 | 15 | 6.7 |

After the death of the nude mice, in-situ tumors were embedded in paraffin and subjected to CD31/PAS immunohistochemical double staining following the same method as described in Example 1. The numbers of MV and VM in the sectioned samples were recorded.

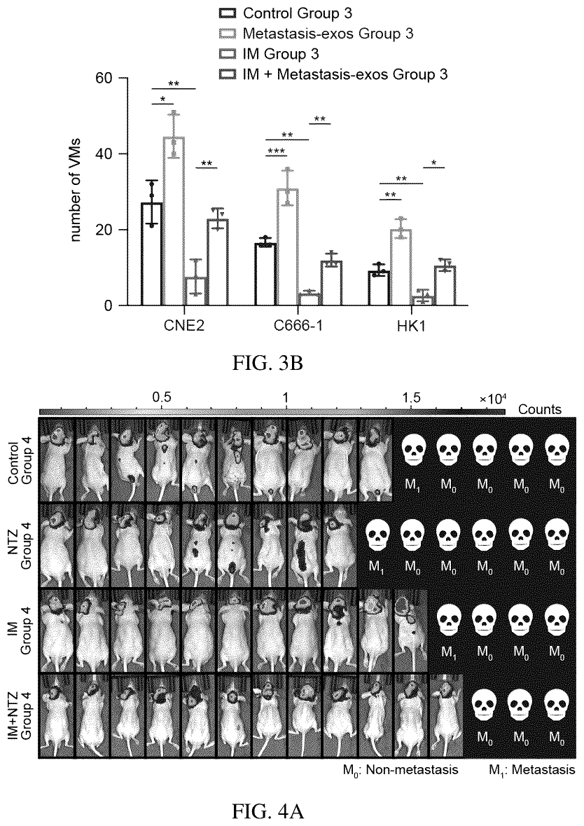

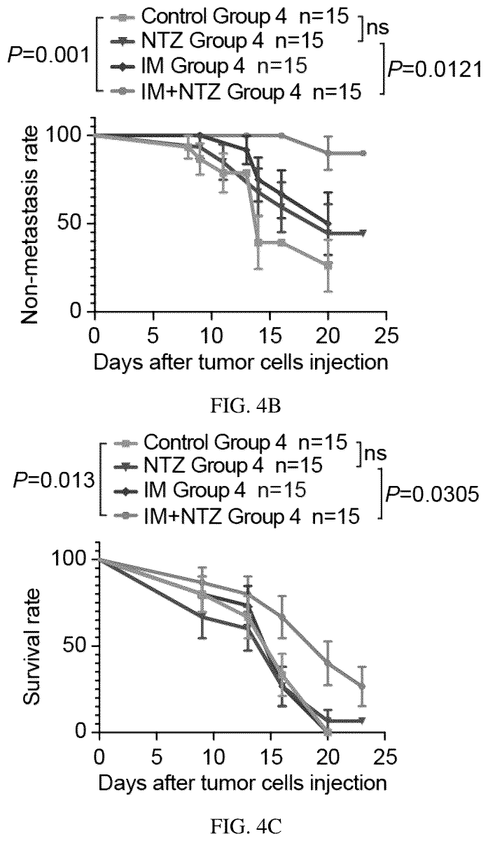

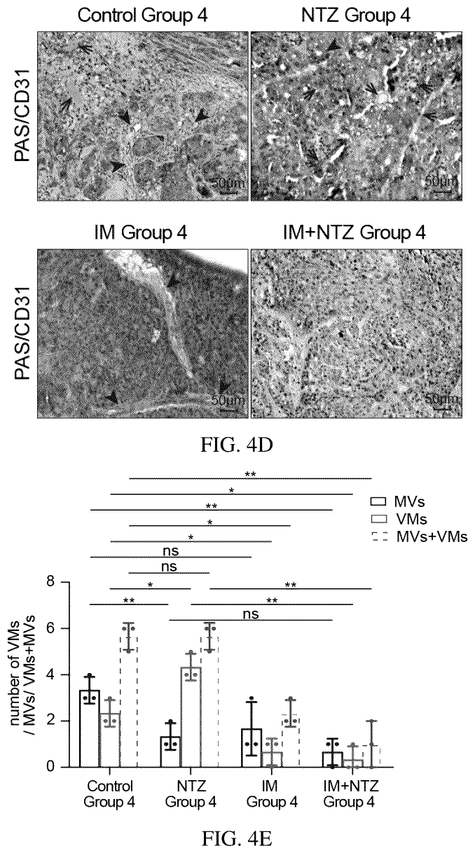

FIG. 4A is whole-body BLI representative images for Control Group 4, NTZ Group 4, IM Group 4, and NTZ+IM Group 4 on day 14 post-tumor inoculation. As demonstrated in FIG. 4A, on day 14 post-inoculation, Control Group 4 exhibited the highest number of nude mice with metastasis, whereas the NTZ+IM Group 4 exhibited the lowest number.

The non-metastasis rate for each group was calculated based on whole-body BLI combined with histological examination of paraffin-embedded sections. The results are presented in FIG. 4B. The non-metastasis rate was calculated according to the following formula:

Non-metastasis Rate (%)=100%−(Number of mice with metastasis/Total number of mice)×100%

FIG. 4B shows the non-metastasis rates over time for Control Group 4, NTZ Group 4, IM Group 4, and NTZ+IM Group 4. Control Group 4 exhibited the lowest non-metastasis rate of 46.7%, whereas NTZ+IM Group 4 demonstrated the highest non-metastasis rate of 60%.

Survival data for each nude mouse were recorded and analyzed using GraphPad Prism software to generate survival curves for each group, as shown in FIG. 4C. This figure indicates that Control Group 4 exhibited the lowest survival rate and the poorest prognosis, while NTZ+IM Group 4 showed the highest survival rate with a statistically significant improvement in prognosis.

FIG. 4D is representative images of CD31/PAS immunohistochemical double staining of tissue sections from NPC in situ tumors in nude mice for Control Group 4, NTZ Group 4, IM Group 4, and NTZ+IM Group 4. Herein, CD31-PAS+structures indicate VM (indicated by normal arrows), while CD31+PAS+structures indicate MV (indicated by black solid arrows).

FIG. 4E is a bar graph of the quantified counts of MV, VM, and MV+VM in tissue sections from NPC in situ tumors in nude mice for Control Group 4, NTZ Group 4, IM Group 4, and NTZ+IM Group 4. As demonstrated in FIG. 4E, Control Group 4 exhibited the highest quantities of MV, VM, and MV+VM, whereas the NTZ+IM Group 4 demonstrated a significant reduction in the numbers of MV, VM, and MV+VM.

The results presented in FIG. 4A to 4E collectively demonstrate that IM significantly inhibits the formation of VM. Furthermore, the combination of IM with NTZ significantly inhibits metastasis of NPC and markedly improves the survival rate in the model nude mice.

The embodiments described above are merely preferred embodiments of this disclosure and are not intended to limit the scope of the disclosure. Any modifications, equivalent substitutions, and improvements made without departing from the fundamental principles of this disclosure shall fall within the protection scope of this disclosure.

What is claimed is:

1. Use of ingenol mebutate (IM) in the preparation of a medicament for the treatment of a tumor, wherein the medicament inhibits the formation of vasculogenic mimicry (VM).

2. The use according to claim 1, wherein the tumor is nasopharyngeal carcinoma (NPC).

3. Use of a combined pharmaceutical composition in the preparation of a medicament for the treatment of a tumor, wherein the combined pharmaceutical composition comprises an anti-angiogenic drug (AAD) and ingenol mebutate (IM).

4. The use according to claim 3, wherein the anti-angiogenic drug (AAD) is nimotuzumab (NTZ).

5. The use according to claim 3, wherein the mass ratio of the anti-angiogenic drug (AAD) to ingenol mebutate (IM) in the combined pharmaceutical composition is 1:1.

6. The use according to claim 3, wherein the medicament for the treatment of a tumor, is for inhibiting tumor metastasis and/or recurrence.

7. The use according to claim 3, wherein the combined pharmaceutical composition comprises two separate formulations administered simultaneously or sequentially.

8. The use according to claim 3, wherein the medicament for the treatment of a tumor further comprises pharmaceutically acceptable excipients.

9. The use according to claim 3, wherein the tumor is nasopharyngeal carcinoma (NPC).

10. The use according to claim 4, wherein the tumor is nasopharyngeal carcinoma (NPC).

11. The use according to claim 5, wherein the tumor is nasopharyngeal carcinoma (NPC).

12. The use according to claim 6, wherein the tumor is nasopharyngeal carcinoma (NPC).

13. The use according to claim 7, wherein the tumor is nasopharyngeal carcinoma (NPC).

14. The use according to claim 8, wherein the tumor is nasopharyngeal carcinoma (NPC).
